(19) United States
(12) Patent Application Publication
(10) Pub. No.: US 2026/0047998 A1
(43) Pub. Date: Feb. 19, 2026

(54) Special Effects Makeup Replicating Mud

(71) Applicant: Joanna Mireau, Vancouver (CA)

(72) Inventor: Joanna Mireau, Vancouver (CA)

(21) Appl. No.: 18/809,287

(22) Filed: Aug. 19, 2024

(51) Int. Cl. A61K 8/96; A61K 8/02; A61K 8/34; A61K 8/92; A61Q 1/02

(52) U.S. Cl. CPC A61K 8/965; A61K 8/0212; A61K 8/345; A61K 8/92; A61Q 1/02; A61K 2800/43

(57) ABSTRACT

A composition including one or more carriers, one or more clays, one or more coloring elements, one or more texturing elements and one or more thickening elements. The composition is safe for skin, hair, prosthetics and in some embodiments, costumes; and accurately replicates the appearance and feel of mud for use in television, film, theatre, or any other medium that requires an actor, or other wearer, to appear muddy.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

The present invention relates generally to the field of special effects makeup of existing art and more specifically relates to a makeup composition that replicates the feel and appearance of mud.

Special effects makeup is an integral part of creating immersive experiences in various forms of entertainment such as theatre, film, and television. The success of special effects makeup lies in its ability to look and feel authentic. In theater, where performances are live and up close, makeup must not only withstand the scrutiny of the audience's gaze but also endure the rigors of stage lighting and movement. Similarly, in film and television, where scenes are captured in high definition and sometimes extreme close-ups, makeup must appear seamless and natural under the scrutiny of the camera lens.

Mud is a substance often portrayed in various forms of media such as TV, theatre, and film to create gritty, earthy effects; however, its use poses significant concerns regarding safety and hygiene. Despite its visual effectiveness in simulating outdoor scenes or adding texture to costumes, applying mud directly onto the actor's skin or hair can lead to numerous health risks. Mud may contain harmful bacteria, parasites, or toxins that can cause skin irritation, infections, or allergic reactions. Additionally, mud is difficult to clean from the skin and hair, potentially causing discomfort and long-term damage. Currently, there are no effective skin and hair safe compositions that effectively simulate mud. Accordingly, a suitable solution is desired.

### SUMMARY OF THE INVENTION

In view of the foregoing disadvantages inherent in the known special effects makeup art, the present disclosure provides special effects makeup replicating mud. The general purpose of the present disclosure, which will be described subsequently in greater detail, is to provide a composition that effectively replicates the feel, appearance, texture and consistency of mud and is safe for application to hair and skin, for use on actors in theatre, television, movies, etc.

A composition replicating mud for special effects makeup is disclosed herein. Generally, the composition may include one or more carriers, one or more clays in an amount sufficient to provide a realistic mud appearance to the composition, one or more texturizing elements in an amount sufficient to provide a realistic mud texture to the composition, one or more coloring elements in an amount sufficient to provide a realistic mud color to the composition, and one or more thickening elements in an amount sufficient to provide a realistic mud consistency to the composition.

According to another embodiment, a composition replicating mud for special effects makeup may include about 45 to 65 wt. % of one or more carriers, about 10 to 15 wt. % of a main solvent, about 10 to 15 wt. % of one or more clays, about 10 to 15 wt. % of one or more texturizing elements, about 5 to 15 wt. % of one or more coloring elements, and about 2 to 8 wt. % of one or more thickening elements.

According to another embodiment, a method of producing a composition that replicates mud for special effects makeup is also disclosed herein. The method may comprise the steps of: mixing, into one or more carriers, a desired amount of one or more clays, the desired amount of the one or more clays chosen based on a desired appearance of the composition; mixing, into the one or more carriers, a desired amount of one or more texturizing elements, the desired amount of the one or more texturizing elements chosen based on a desired texture of the composition; mixing, into the one or more carriers, a desired amount of one or more coloring elements, the desired amount of the one or more coloring elements chosen based on a desired color of the composition; and mixing, into the one or more carriers, a desired amount of one or more thickening elements, the desired amount of the one or more thickening elements chosen based on a desired consistency of the composition.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF FIGURES

The figures which accompany the written portion of this specification illustrate embodiments and methods of use for the present disclosure, special effects makeup replicating mud, constructed and operative according to the teachings of the present disclosure.

FIG. 1 is a perspective view of a composition replicating mud having been applied to hair, face and body of a wearer, according to an embodiment of the disclosure.

FIG. 2 is a front view of the composition applied to the hair, face and body of the wearer, according to an embodiment of the disclosure.

FIG. 3 is a table illustrating ingredients of the composition and example amounts, according to an embodiment of the present disclosure.

FIG. 4 is another table illustrating ingredients of the composition and example amounts, according to an embodiment of the present disclosure.

FIG. 5 is another table illustrating ingredients of the composition and example amounts, according to an embodiment of the present disclosure.

FIG. 6 is another table illustrating ingredients of the composition and example presence by weight in the composition, according to an embodiment of the present disclosure.

FIG. 7 is another table illustrating ingredients of the composition and example presence by weight in the composition, according to an embodiment of the present disclosure.

FIG. 8 is another table illustrating ingredients of the composition and example presence by weight in the composition, according to an embodiment of the present disclosure.

FIG. 9 is a flow diagram illustrating a method of producing a composition that replicates mud for special effects makeup, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to special effects makeup and more particularly to special effects makeup replicating mud. Generally disclosed is a composition that is safe for skin, hair, prosthetics and in some embodiments, costumes, and accurately replicates the appearance and feel of mud. The composition may be adjusted, depending on desired color, opacity and/or texture and can be matched to any real mud. The composition is able to be used in film, television, theatre, or any other medium requiring an actor or other wearer to look muddy. The composition can be applicable to makeup, hair and prosthetic departments.

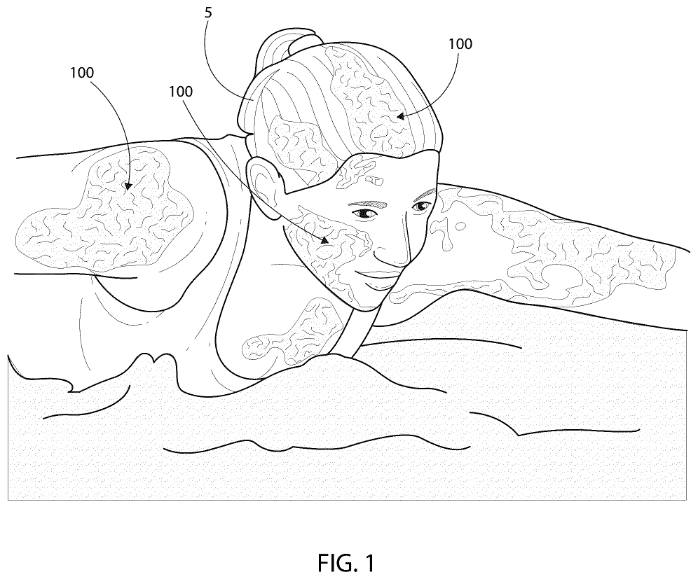

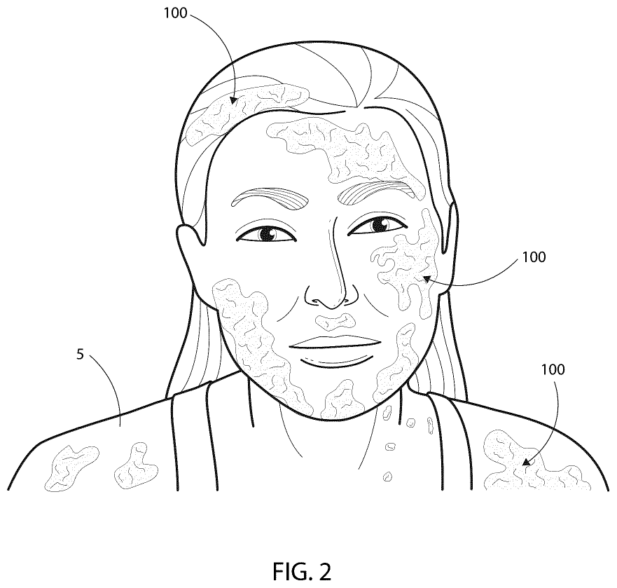

Referring first to FIGS. 1-2, there is shown a composition 100 applied to hair, face and body of a wearer 5, according to one or more embodiments of the present disclosure. As above, the composition 100 preferably looks and feels just like real mud. For example, FIG. 1 illustrates the composition 100 having been mixed to appear similar to a mud pit the wearer 5 is shown crawling through. The composition 100 is safe for the skin of the wearer 5 and hair of the wearer 5, thereby providing the visual effects of mud, whilst being safe and nontoxic to the wearer 5.

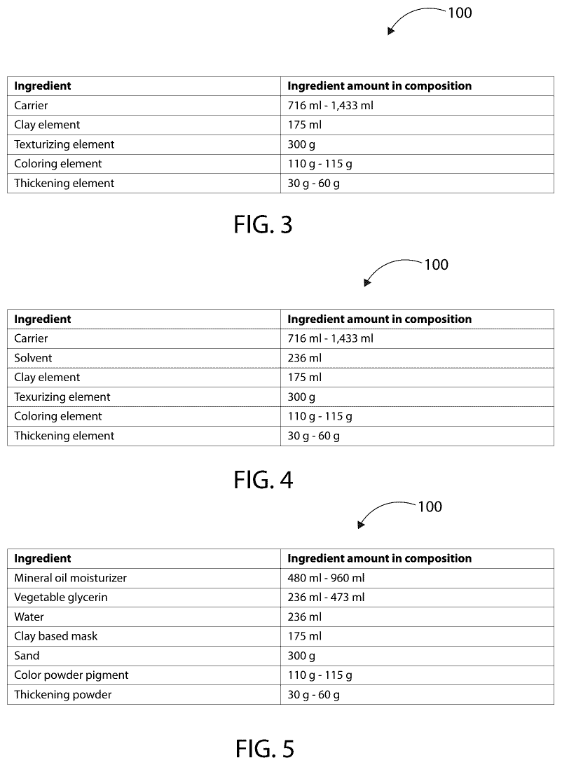

Referring now also to FIGS. 3-8, illustrating tables demonstrating ingredients of the composition 100 and example amounts within the composition 100, according to one or more embodiments of the present disclosure. As shown in FIG. 3, in some embodiments, the composition 100 may include one or more carriers, one or more clays, one or more coloring elements, one or more texturizing elements and one or more thickening elements. Further, as shown in FIG. 4, a main solvent may be provided within the composition 100 to enable efficient mixing of the ingredients within the composition 100. For example, the main solvent may be water, as shown in FIG. 5.

In some embodiments, the one or more carriers may include an emollient based ingredient. For example, the one or more carriers may include mineral oil. In some embodiments, the mineral oil may be provided as a part of a mineral oil based moisturizer. For example, LUBRIDERM® may be used as the carrier, or one of the carriers, in the composition 100. In particular, the mineral oil based moisturizer may include (but is not limited to including) the mineral oil, water, capris triglyceride, stearic acid, cetyl alcohol, parabens, and pantheonol.

In some embodiments, vegetable glycerin may also be provided as one of the carriers of the composition 100. The vegetable glycerin may also provide emollient and humectant properties to the composition 100, enabling the composition 100 to moisturize and prevent drying of the skin or hair of the wearer 5 and aiding in removal of the composition 100 form the wearer 5. The glycerin may further act as a solvent in the composition 100 to aid in mixing of the composition 100.

The thickening element may be used to provide a desired realistic mud consistency to the composition 100. Accordingly, an amount of the thickening element may be adjusted depending on desired consistency of the composition 100. For example, if a scene requires a thick mud, more thickening element would be added to the composition 100 than would be added for a scene requiring a thinner mud. The water may also aid in thinning down the composition 100.

In some embodiments, the thickening element may include a powder. For example, the thickening element may be ULTRAMUD®. It should however be appreciated that other thickening elements may be used instead of, or in addition to, the ULTRAMUD®. For example, xantham gum, guar gum, cellulose gum, cetyl alcohol, sodium alginate, or the like. It should be appreciated that this list is not exhaustive; and also, that the thickening element is not limited to any elements listed here.

In some embodiments, the one or more clays may be provided to add a realistic mud appearance to the composition 100; as well as a realistic texture. Particularly, the one or more clays 120 may dry when applied to the skin and/hair of the wearer 5 to replicate dried mud on the skin/hair. In some embodiments, the one or more clays may include kaolin clay and/or bentonite clay. However, other clays 120 may also be used in the composition 100, instead of, or in addition to the kaolin clay and/or bentonite clay. The clay chosen and the amount of clay may depend on the type of mud needed. For example, if a drier mud composition 100 is needed, more clay may be added, bentonite clay may be chosen over kaolin clay, etc.

In some embodiments, the one or more clays may be provided within a clay based composition, such as a clay based mask. In some examples, the clay based mask may include the one or more clays, humectant, mineral oxide, thickener, and emollient.

The one or more coloring elements are chosen to provide a realistic mud color to the composition 100. The mud color may depend on what is required for the particular scene, and as such, a color of the coloring element is chosen based on the desired color of the overall composition 100. The one or more coloring elements may also be mixed together to create the desired color. In some embodiments, the one or more coloring elements may include powder pigment. For example, the one or more coloring elements may be one or more FLEET STREET DIRTWORKS® powders.

The texturizing element may be provided to add a realistic mud texture to the composition 100. In some examples, the texturizing element may include sand, such as (but not limited to) ballast grey sand, to replicate the particles present in mud.

In some embodiments, a prepared mixture of the composition 100 may yield approximately 1.5 liters. It should however be appreciated that the composition 100 is not limited to a particular amount. In the embodiment yielding 1.5 liters, the composition 100 may include (but is not limited to) between about 480 ml to about 960 ml, about 236 ml of said water, between about 236 ml to about 473 ml of the vegetable glycerin, between about 30 g to about 60 g of the thickening powder, between about 110 g to about 115 g of the powder pigment, about 175 ml of the clay based mask and about 300 g of the sand.

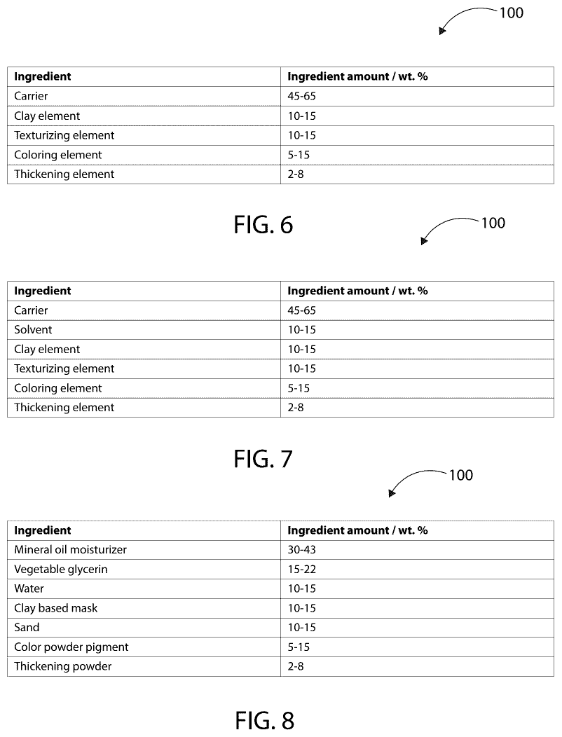

Referring now also to FIGS. 6-8, in some embodiments, the composition 100 may include about 45 to 65 wt. % of the one or more carriers, about 15 to 10 wt. % of the main solvent, about 2 to 8 wt. % of the thickening element, about 10 to 15 wt. % of the one or more clays, about 10 to 5 wt. % of the one or more coloring elements, and about 5 to 15 wt. % of the texturizing element. Again, as above, the one or more carriers may include mineral based moisturizer and vegetable glycerin. In this embodiment, the mineral based moisturizer may be present in about 30 to 43 wt. % and the vegetable glycerin may be present in about 15 to 22 wt. %. Further, the clay based mask may be present in about 10 to 15 wt. %; the water may be present in about 10 to 15 wt. %; the thickening powder may be present in about 2 to 8 wt. %; the powder pigment may be present in about 5 to 15 wt. %; and the sand may be present in about 10 to 15 wt. %.

As has been discussed above, amounts of each of the ingredients may be adjusted depending on desired texture, color, consistency, viscosity, feel, appearance, etc. of the mud composition 100. The amounts and % wt. may also vary based on a density of the ingredients used in the composition 100. As such, it should be appreciated that the ingredients listed here are not limited to the exact measurements provided above, as adjustments can be made based on user preference. It should be appreciated that the above amounts are included to provide instructions for someone knowledgeable in the art to replicate the mud composition 100.

It should also be appreciated that each ingredient of the composition 100 may contribute more than one characteristic of the composition 100 and is not limited to the particular characteristic called out in the claims and throughout this specification; and that each ingredient is added to contribute to an overall composition 100 replicating mud. For example, the clays may contribute to the texture as well as the mud appearance, the coloring element may additionally contribute to the consistency as well as the color, and the like.

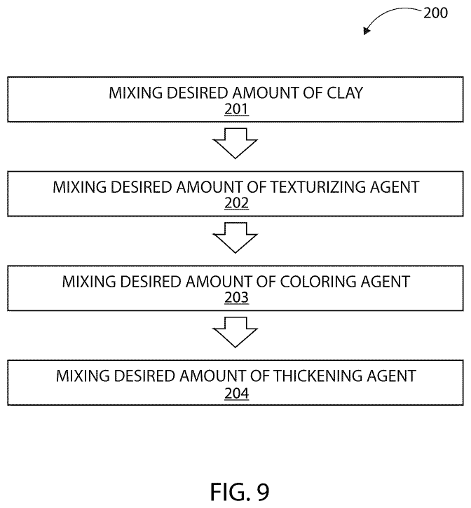

Referring now to FIG. 9, showing a flow diagram illustrating a method of producing a composition replicating mud (“method” 200), according to one or more embodiments of the present disclosure. In particular, the method 200 may include one or more components or features of the composition 100 as described above. As illustrated, the method 200 may include the step of: step one 501, mixing a desired amount of one or more clays into one or more carriers, the desired amount of the one or more clays chosen based on a desired appearance of the composition; step two 202, mixing a desired amount of one or more texturizing elements into the one or more carriers, the desired amount of the one or more texturizing elements chosen based on a desired texture of the composition; step three 203, mixing a desired amount of one or more coloring elements into the one or more carriers, the desired amount of the one or more coloring elements chosen based on a desired color of the composition; and step four 204, mixing a desired amount of one or more thickenings element into the one or more carriers, the desired amount of the one or more thickenings elements chosen based on a desired consistency of the composition.

It should be noted that certain steps may be optional and may not be implemented in all cases. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for producing the composition are taught herein.

It should be understood by one of skill in the art that the disclosed invention is described here in a few exemplary embodiments of many. No particular terminology or description should be considered limiting on the disclosure or the scope of any claims issuing therefrom.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the relevant patent offices and the public generally, and especially the scientist, engineers and practitioners in the art who are not familiar with patent or legal terms or phraseology, to determine quickly from a cursory inspection the nature and essence of the technical disclosure of the application.

1. A composition replicating mud for special effects makeup, the composition comprising:
one or more carriers;
one or more clays in an amount sufficient to provide a realistic mud appearance to the composition;
one or more texturizing elements in an amount sufficient to provide a realistic mud texture to the composition;
one or more coloring elements in an amount sufficient to provide a realistic mud color to the composition; and
one or more thickening elements in an amount sufficient to provide a realistic mud consistency to the composition.

2. The composition of claim 1, further comprising a main solvent for the composition, and wherein the main solvent is water.

3. The composition of claim 2, wherein the composition comprises about 10 to 15 wt. % of the water.

4. The composition of claim 3, wherein the one or more carriers includes mineral oil.

5. The composition of claim 4, wherein the mineral oil is provided as a part of a mineral oil based moisturizer.

6. The composition of claim 5, wherein the composition comprises between about 30 to 43 wt. % of the mineral oil based moisturizer.

7. The composition of claim 6, wherein the one or more carriers further includes vegetable glycerin.

8. The composition of claim 7, wherein the composition comprises between about 15 to 22 wt. % of the vegetable glycerin.

9. The composition of claim 8, wherein the one or more thickening elements includes a thickening powder.

10. The composition of claim 9, wherein the composition comprises about 2 to 8 wt. % of the thickening powder.

11. The composition of claim 10, wherein the one or more coloring elements includes a powder pigment.

12. The composition of claim 11, wherein the composition comprises about 5 to 15 wt. % of the powder pigment.

13. The composition of claim 12, wherein the one or more clays are provided as a part of a clay based mask.

14. The composition of claim 13, wherein the composition comprises about 10 to 15 wt. % of the clay based mask.

15. The composition of claim 14, wherein the one or more texturizing elements includes sand.

16. The composition of claim 15, wherein the sand is ballast grey sand.

17. The composition of claim 16, wherein the composition comprises about 10 to 15 wt. % of the sand.

18. A composition replicating mud for special effects makeup, the composition comprising:
about 45 to 65 wt. % of one or more carriers;
about 10 to 15 wt. % of a main solvent;
about 10 to 15 wt. % of one or more clays;
about 10 to 15 wt. % of one or more texturizing elements;
about 5 to 15 wt. % of one or more coloring elements; and
about 2 to 8 wt. % of one or more thickening elements.

19. The composition of claim 18, wherein the one or more carriers includes about 30 to 43 wt. % of a mineral oil based moisturizer and about 15 to 22 wt. % of a vegetable glycerin;
wherein the main solvent includes water;
wherein the one or more clays includes a clay based mask;
wherein the one or more texturizing elements includes sand;
wherein the one or more coloring elements includes powder pigment; and
wherein the one or more thickening elements includes thickening powder.

20. A method of producing a composition that replicates mud for special effects makeup, the method comprising the steps of:
mixing, into one or more carriers, a desired amount of one or more clays, the desired amount of the one or more clays chosen based on a desired appearance of the composition;
mixing, into the one or more carriers, a desired amount of one or more texturizing elements, the desired amount of the one or more texturizing elements chosen based on a desired texture of the composition;
mixing, into the one or more carriers, a desired amount of one or more coloring elements, the desired amount of the one or more coloring elements chosen based on a desired color of the composition; and
mixing, into the one or more carriers, a desired amount of one or more thickening elements, the desired amount of the one or more thickening elements chosen based on a desired consistency of the composition.
